(19) United States
(12) Patent Application Publication
(10) Pub. No.: US 2006/0210620 A1
(43) Pub. Date: Sep. 21, 2006

(54) Co-precipitated amorphous losartan and dosage forms comprising the same

(76) Inventors: Pannanchukunnath Manoj Kumra, New Delhi (IN); Ramalingam Manikandan, Tamil Nadu (IN); Romi Barat Singh, Uttar Pradesh (IN); Vishnubhotla Nagaprasad, Andhra Pradesh (IN)

Correspondence Address: RANBAXY INC., 600 COLLEGE ROAD EAST, SUITE 2100, PRINCETON, NJ, 08540, US

(21) Appl. No.: 10/542,587

(22) PCT Filed: Jan. 21, 2004
(86) PCT No.: PCT/IB04/00144

§ 371 Date: Apr. 7, 2006

(30) Foreign Application Priority Data
Jan. 21, 2003 (IN) 54/DEL/2003

(51) Int. Cl. A61K 31/41; A61K 9/48; A61K 9/20

(52) U.S. Cl. 424/451; 424/464; 514/381

(57) ABSTRACT

The technical field of the invention relates to spray dried, co-precipitate amorphous losartan dosage forms that are stable over time and processes for their preparation. The processes stabilize the amorphous losartan. The process includes preparing an aqueous solution of losartan and one or more hydrophilic polymers; and spray drying the aqueous solution of losartan and one or more hydrophilic polymers to form a mixture. The amorphous losartan and one or more hydrophilic polymers are co-precipitated from the aqueous solution.

### FIELD OF THE INVENTION

The technical field of the invention relates to spray dried, co-precipitated amorphous losartan dosage forms that are stable over time and processes for their preparation.

### BACKGROUND OF THE INVENTION

Losartan is in a new class of antihypertensive agents which inhibit the action of the vasopressor hormone angiotensin II. It thereby helps in combating angiotensin-induced hypertension. Chemically, losartan is 2-butyl-4-chloro-1-[2′-(1H-tetrazol-5-yl)-biphenyl-4-yl)methyl]-5-(hydroxymethyl) imidazole, and is commercially available from Merck as Cozaar® in 25 mg, 50 mg, and 100 mg tablets. Losartan can be combined with diuretics, particularly thiazides, for the treatment of hypertension, but also is useful for the treatment of congestive heart failure. Losartan is known to exist in both crystalline and amorphous forms; the amorphous form being the preferred form in pharmaceutical compositions. However, amorphous solids by their nature are high-energy forms, hence may be thermodynamically unstable and quickly convert to the crystalline form. Thus, in order to exploit any advantages of the amorphous solid forms, their conversion to the crystalline form needs to be stopped or slowed down for at least a reasonable period of time.

U.S. Pat. No. 4,127,647 discloses a process of preparing stable amorphous solids of macrolide antibiotics, the process comprising spray drying a solution in a volatile organic solvent of a macrolide antibiotic and at least one cellulose polymer. Following such a process for losartan, however, will lead to an amorphous solid having traces of organic solvents entrapped into it, which may not be pharmaceutically acceptable.

U.S. Pat. No. 5,608,075 is listed in the U.S. Food and Drug Administration's Orange Book for Losartan. This patent claims Form I and Form II losartan and a process for preparing Form II losartan by heating Form I losartan. X-ray diffraction angles and differential scanning calorimetry data for Forms I and II losartan are provided in the patent.

### SUMMARY OF THE INVENTION

In one general aspect there is provided a process for stabilizing amorphous losartan. The process includes preparing an aqueous solution of losartan and one or more hydrophilic polymers; and spray drying the aqueous solution of losartan and one or more hydrophilic polymers to form a mixture. The amorphous losartan and one or more hydrophilic polymers are co-precipitated from the aqueous solution.

Embodiments of the process may include one or more of the following features. For example, the aqueous solution may be prepared in an aqueous solvent selected from the group consisting of water, water miscible solvents and mixtures thereof and, in particular, may be water. The water miscible solvent may include one or more of methanol, ethanol, n-propanol and isopropanol.

The hydrophilic polymer may be one or more of polyvinylpyrrolidone (PVP), polyvinyl alcohol, hydroxypropyl methylcellulose, methylcellulose, carboxymethyl cellulose, sodium carboxymethylcellulose, hydroxyethylcellulose, polyvinyl acetate, carbopols and combinations thereof and, in particular, the hydrophilic polymer may be polyvinylpyrrolidone. The polyvinylpyrrolidone comprises one or more of the grades PVP K-15, K-25, K-30, K-60 and K-90 and, in particular, may be PVP K-30. The ratio of polyvinylpyrrolidone to losartan may be from about 0.5:1 to about 1:1.5 and, in particular, may be 1:1.

The hydrophilic polymer may be polyvinyl alcohol or hydroxypropyl methylcellulose.

The spray drying may be carried out at a temperature of more than about 60° C. and, in particular, the spray drying may be carried out at a temperature of about 135° C.

The process may further include processing the mixture with one or more pharmaceutically inert excipients. The pharmaceutically inert excipients may be one or more diluents, binders, disintegrants, coloring agents, flavoring agents, stabilizers, surfactants, lubricants, glidants, plasticizers, and preservatives. The process may further include forming one or more of a tablet, a capsule, and a powder.

The losartan may according to the process may remain amorphous as measured by X ray diffraction after accelerated stability testing at 40° C. and 75% relative humidity for three months.

In another general aspect there is provided a pharmaceutical composition that includes a co-precipitated mixture of amorphous losartan and one or more hydrophilic polymers.

Embodiments of the pharmaceutical composition may include one or more of the following features. For example, the losartan may remain amorphous as measured by X ray diffraction after accelerated stability testing at 40° C. and 75% relative humidity for three months. The hydrophilic polymer may be one or more of polyvinylpyrrolidone (PVP), polyvinyl alcohol, hydroxypropyl methylcellulose, methylcellulose, carboxymethyl cellulose, sodium carboxymethylcellulose, hydroxyethylcellulose, polyvinyl acetate, carbopols and combinations thereof and, in particular, may be polyvinylpyrrolidone.

The pharmaceutical composition may further include one or more pharmaceutically inert excipients. The one or more pharmaceutically inert excipients may be one or more of diluents, binders, disintegrants, coloring agents, flavoring agents, stabilizers, surfactants, lubricants, glidants, plasticizers and preservatives.

The pharmaceutical composition may be a solid dosage form and the solid dosage form comprises one or more of tablets, capsules, and powders, and, in particular, may be a tablet.

The ratio of polyvinylpyrrolidone to losartan may be from about 0.5:1 to about 1:1.5 and, in particular, may be 1:1.

In another general aspect there is provided a method for the treatment of angiotensin-induced hypertension in a mammal. The method includes administering a pharmaceutical composition that includes a co-precipitate of amorphous losartan and one or more hydrophilic polymers, and one or more pharmaceutically inert excipients.

Embodiments of the method may include one or more of the following features. For example the hydrophilic polymer may be polyvinylpyrrolidone, the polyvinylpyrrolidone may be one or more of the grades PVP K-15, K-25, K-30, K-60 and K-90, and, in particular, the polyvinylpyrrolidone grade may be PVP K-30. The ratio of polyvinylpyrrolidone to losartan may be from about 0.5:1 to about 1:1.5 and, in particular, may be 1:1.

The losartan remains amorphous as measured by X ray diffraction after accelerated stability testing at 40° C. and 75% relative humidity for three months.

In another general aspect there is provided a co-precipitate of amorphous losartan and one or more hydrophilic polymers. Embodiments of the co-precipitate may include any one or more of the following features or the features described above. For example, the ratio of hydrophilic polymer to losartan may be from about 0.5:1 to about 1:1.5 and, in particular, may be 1:1.

The details of one or more embodiments of the inventions are set forth in the description below. Other features, objects and advantages of the inventions will be apparent from the description and claims.

### DESCRIPTION OF THE DRAWINGS

FIG. 1 is a set of X-ray diffraction patterns for Example 1.

FIG. 2 is a set of X-ray diffraction patterns for Example 2 showing the initial measurement and over time.

FIG. 3 is an X-ray diffraction patterns for Example 3 as measured initially.

FIG. 4 is an X-ray diffraction patterns for Example 3 as measured after one month of accelerated stability testing.

FIG. 5 is an X-ray diffraction patterns for Example 3 as measured after three months of accelerated stability testing.

FIG. 6 is an X-ray diffraction patterns for Example 4 as measured initially.

FIG. 7 is an X-ray diffraction patterns for Example 4 as measured after one month of accelerated stability testing.

FIG. 8 is an X-ray diffraction patterns for Example 4 as measured after three months of accelerated stability testing.

FIG. 9 is a set of X-ray diffraction patterns for Example 5 showing the initial measurement and over time.

### DETAILED DESCRIPTION OF THE INVENTION

Amorphous losartan is highly hygroscopic and quickly absorbs moisture to spontaneously convert to crystalline form. It has now been found that the hydrophilic polymers of the present invention help in forming a solid dispersion with losartan, thereby reducing its affinity for moisture. The resulting amorphous solid is stable and does not convert to crystalline form after accelerated stability studies after three months.

The term “losartan” as used herein includes free losartan as well as any of its pharmaceutically acceptable salts thereof. Some of the pharmaceutically acceptable salts of losartan are salts with sodium, potassium, magnesium, calcium and the like. In particular, for its physiological acceptability, losartan potassium may be used.

The term “stable” as used herein refers to less than about 5% conversion of the amorphous form of losartan to a crystalline form of losartan when stored at 40° C. and 75 percent relative humidity for three months.

In one embodiment, stable amorphous losartan may be prepared by a process that includes forming a solution of hydrophilic polymer in an aqueous solvent, adding losartan into the aqueous solution, and removing the solvent by spray drying, thereby co-precipitating the amorphous losartan and hydrophilic polymers. In another embodiment, stable amorphous losartan may be prepared by a process that includes forming a solution of losartan in an aqueous solvent, adding hydrophilic polymer into the aqueous solution, and removing the solvent by spray drying, again, thereby co-precipitating the amorphous losartan and hydrophilic polymers.

The spray dryer used for drying the aqueous solution may be any of the conventional spray driers known in the art including nozzle type, disc type or jet type. Based on the selection of spray dryer, the various process parameters may be varied. In particular, the spray rate and air pressure may vary in the range of about 1 ml/min to about 50 ml/min and about 1 kg/m² to about 2 kg/m², respectively. The drying temperature in the spray dryer must be greater than 60° C., and in particular, between about 120° C. and about 250° C.

Suitable aqueous solvents should be capable of dissolving both losartan and hydrophilic polymers and be chemically inert with respect to both. Further, the solvent needs to be sufficiently volatile at temperatures below the degradation temperature of the components in the solution. Examples of suitable aqueous solvents include water, water miscible solvents, and mixtures thereof. The water miscible solvents may include lower aliphatic alcohols such as one or more of methanol, ethanol, n-propanol, isopropanol, and the like. The amount of aqueous solvent used should be an amount which is sufficient enough to produce a consistent, easily sprayed mixture through the nozzle but yet not so much that it does not exhibit proper drying. In particular, the total concentration of losartan and hydrophilic polymer in the solution may be less than about 50% by weight of the total volume of the solution.

Examples of hydrophilic polymers may include one or more of polyvinylpyrrolidone (PVP), polyvinyl alcohol, hydroxypropyl methylcellulose, methylcellulose, carboxymethyl cellulose, sodium carboxymethylcellulose, hydroxyethylcellulose, polyvinyl acetate, carbopols and combinations thereof. In particular, polyvinylpyrrolidone may be used. The average molecular weight of polyvinylpyrrolidone may vary from about 10,000 to about 360,000. It is commercially available in five viscosity grades identified by their K-value: K-15, K-25, K-30, K-60 and K-90, according to viscosity in ascending order. The ratio of polyvinylpyrrolidone to losartan may vary from about 0.5:1 to about 1.5:1, depending upon the grade of polyvinylpyrrolidone selected.

Stable amorphous losartan prepared as generally described above may be further processed with one or more pharmaceutically inert excipients to prepare pharmaceutical compositions. The term “pharmaceutical composition” includes solid dosage forms such as one or more of tablets, capsules, and powders that are formulated by conventional methods of admixture such as one or more of blending, filling, and granulation. Of course, other formulation methods also may be used. The dosage form may be optionally coated with one or more film forming polymers.

In one embodiment, the losartan tablet may be prepared by blending a spray dried, co-precipitated mixture of losartan potassium and hydrophilic polymer with diluents and disintegrants, mixing the blend with lubricant and glidants, directly compressing the mixed blend in a suitable tableting machine, and coating with one or more film forming polymers.

In alternative embodiments, dry granulation and wet granulation techniques may be used for preparing losartan tablets.

Coating may be performed by applying one or more film forming polymers with or without other pharmaceutically inert excipients. This may be done as a solution or suspension using any conventional coating technique known in the prior art, such as spray coating in a conventional coating pan or fluidized bed processor, or dip coating.

Suitable film forming polymers include one or more of ethylcellulose, hydroxypropyl methylcellulose, hydroxypropyl cellulose, methylcellulose, carboxymethylcellulose, hydroxymethylcellulose, hydroxyethylcellulose, cellulose acetate, hydroxypropyl methylcellulose phthalate, cellulose acetate phthalate, cellulose acetate trimellitate, waxes, methacrylic acid polymers such as Eudragit® RL and RS, and mixtures thereof. The coating can also be performed using any commercially available ready to coat preparations such as opadry-AMB, opadry-white, opadry-clear, etc.

Suitable solvents used for making a solution/suspension of film forming polymer include one or more of methylene chloride, isopropyl alcohol, acetone, methanol, ethanol, water and mixtures thereof.

In another embodiment, losartan capsules may be prepared by blending the spray dried, co-precipitated mixture of losartan and hydrophilic polymer with other pharmaceutically inert excipients and filling into suitably sized hard gelatin capsules.

The term “pharmaceutically inert excipient” as used herein includes one or more of diluents, binders, disintegrants, coloring agents, flavoring agents, stabilizers, surfactants, lubricants/glidants, plasticizers and preservatives for pharmaceutical compositions.

In yet another embodiment, the spray dried, co-precipitated mixture of losartan and hydrophilic polymer may be dissolved or dispersed into a suitable carrier and filled into soft gelatin capsules using conventional techniques known in the art. Suitable examples of carriers for use in soft gelatin capsules include one or more of soyabean oil, cottonseed oil, olive oil and the like.

The amorphous solids of the following examples were evaluated for the presence of crystals and/or other degradative products using X-ray diffraction (XRD) techniques, supplemented with infrared and differential scanning calorimetry. The examples are meant to further exemplify the invention and are not intended to limit the scope of the invention.

### EXAMPLE 1

100 gm of crystalline losartan potassium were dissolved in 1000 ml of water. The solution obtained was dried and co-precipitated at 135° C. at spray rate of 5.0 ml/min and at a pressure of 1.5 kg/cm² in a spray drier for about 30 minutes. The amorphous solid thus obtained was used as a control.

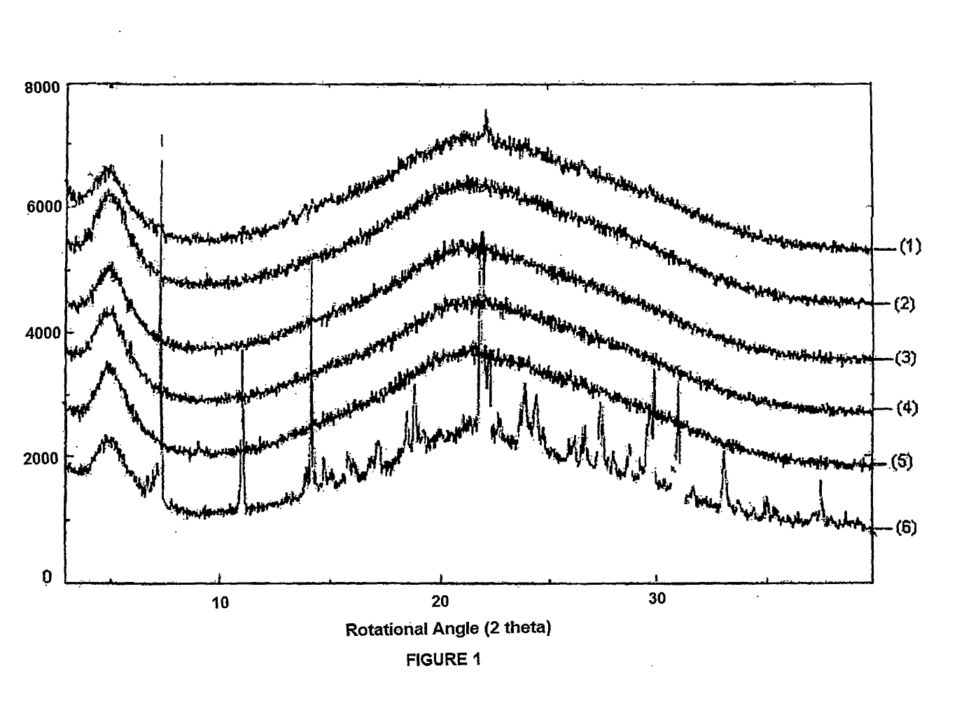

When the control was subjected to accelerated stability conditions at 40° C. and 75% relative humidity, almost all of the solid material converted to the crystalline form, as evident from the XRD spectra in FIG. 1. In FIG. 1, spectra (1)-(6) are for the spray dried, co-precipitated mix with spectra (1)-(5) taken initially after co-precipitating and spectra (6) taken later in time. Specta (1)-(5) show that the losartan is initially amorphous and spectra (6) shows that the losartan is converted to a crystalline form in a short time, as is known in the art.

### EXAMPLE 2

100 gm of crystalline losartan potassium and 50 gm of polyvinylpyrrolidone K-30 were dissolved in 1000 ml of water. The solution obtained was dried at 135° C. at a spray rate of 5.0 ml/min and at a pressure of 1.5 kg/cm² in a spray drier for about 30 minutes. The amorphous solid was obtained by co-precipitation.

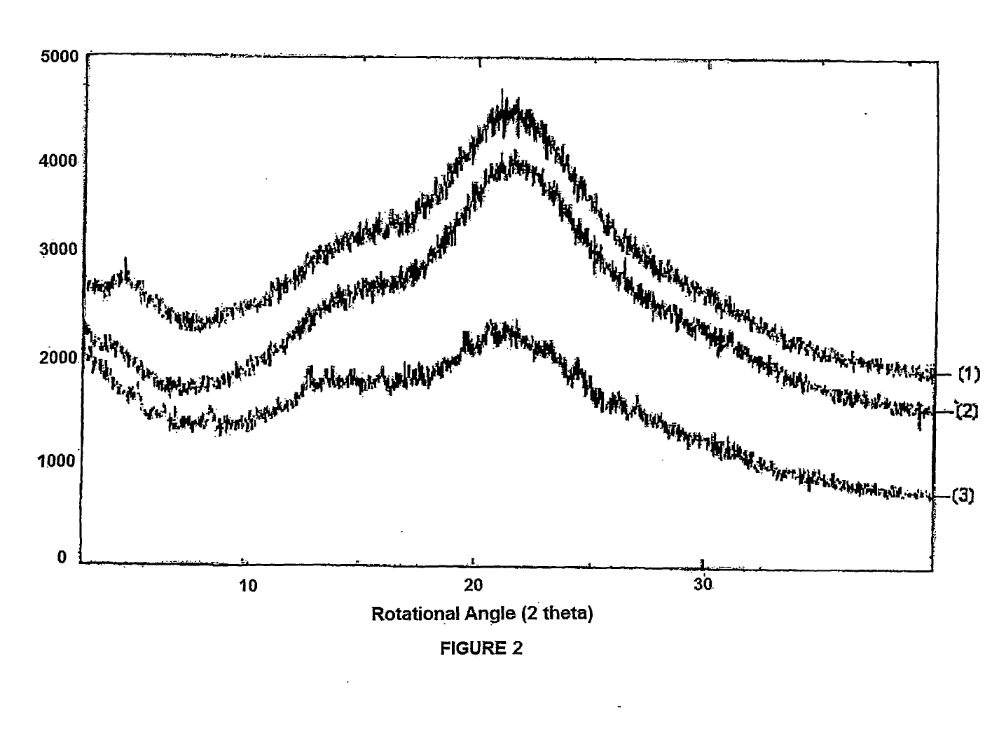

When the amorphous solid obtained was subjected to accelerated stability conditions at 40° C. and 75% relative humidity, it remained in the amorphous form as evident from the XRD spectra in FIG. 2. In FIG. 2, spectra (1) is for the spray dried, co-precipitated mix, spectra (2) is for the spray dried, co-precipitated mix after 14 hours storage at room temperature, and spectra (3) is for the spray dried, co-precipitated mix after 24 hours storage at 40° C. and 75% relative humidity, which is believed to be sufficient time to show that conversion to crystalline does not occur.

### EXAMPLE 3

100 gm of crystalline losartan potassium and 50 gm of polyvinylpyrrolidone K-30 were dissolved in 1000 ml of water. The solution obtained was dried at 135° C. at a spray rate of 5.0 ml/min and at a pressure of 1.5 kg/cm² in a spray drier for about 30 minutes. The amorphous solid was obtained by co-precipitation.

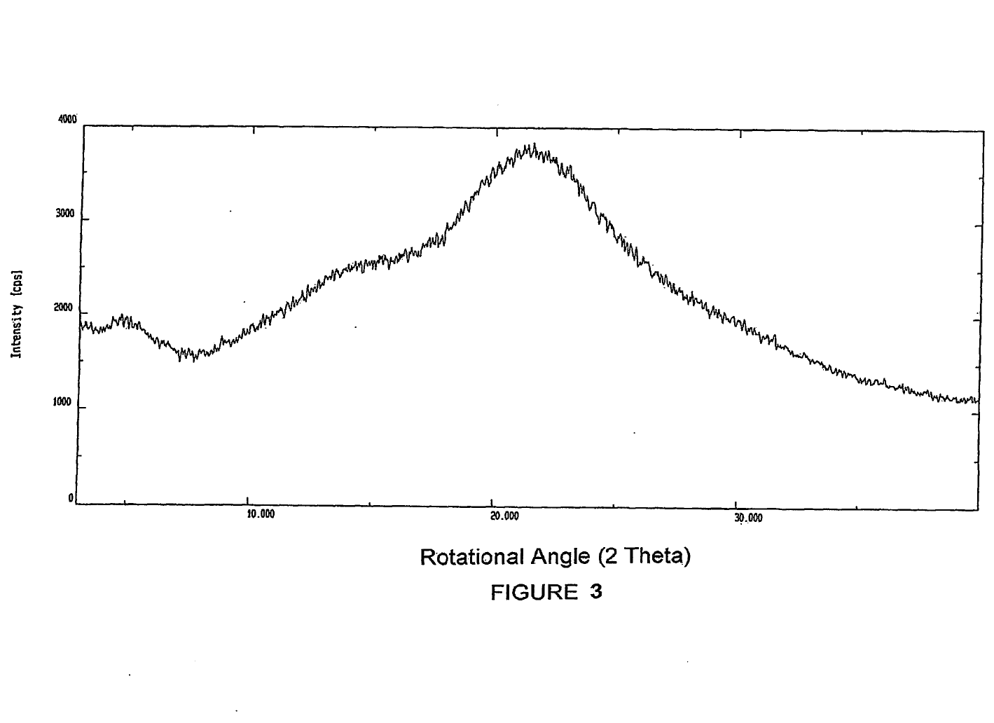

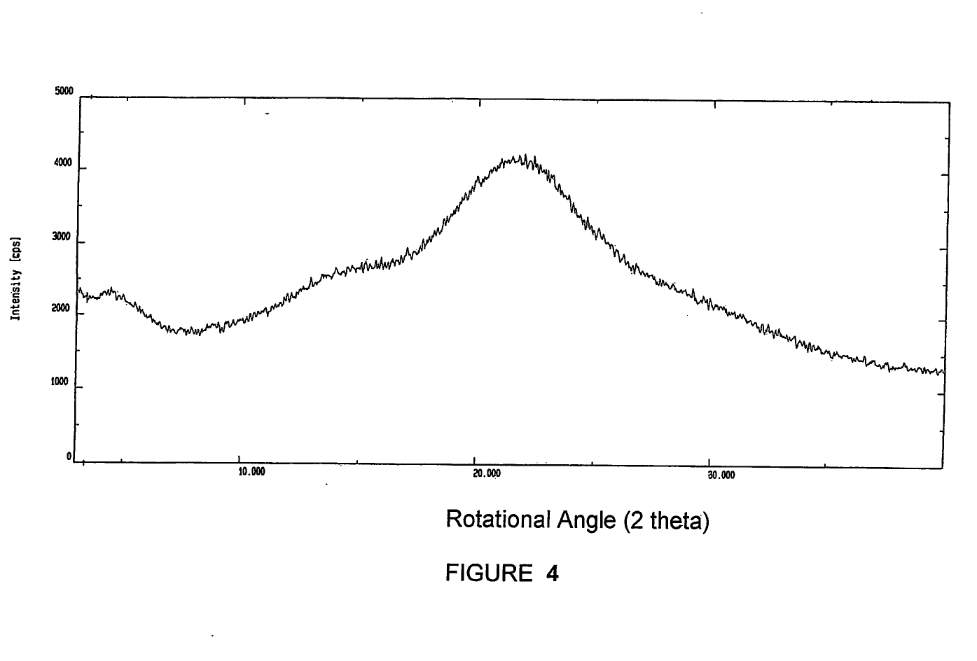

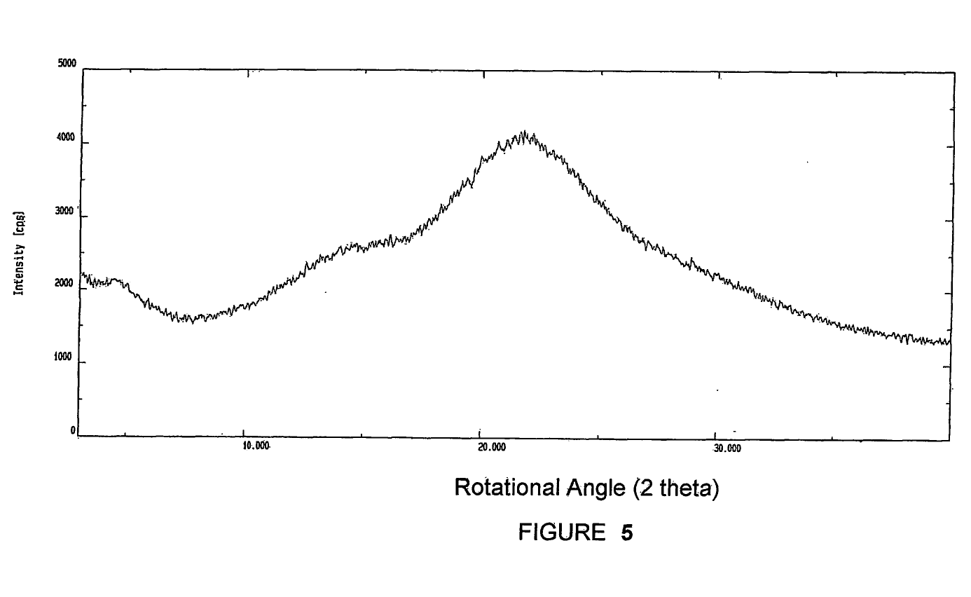

The amorphous, co-precipitated solid obtained was subjected to accelerated stability conditions at 40° C. and 75% relative humidity for three months. It remained in the amorphous form for the three months of accelerated stability testing as evident from the XRD spectra in FIGS. 3-5. The spectra of FIG. 3 is that of the spray dried, co-precipitated mix as measured initially after co-precipitation. This spectra shows the amorphous form of losartan. The spectra of FIG. 4 is that of the spray dried, co-precipitated mix as measured after one month of storage at the accelerated stability conditions (i.e., 40° C. and 75% relative humidity). This spectra shows that the losartan remains in the amorphous form. The spectra of FIG. 5 is that of the spray dried, co-precipitated mix as measured after three months of storage at the accelerated stability condition. Again, this spectra shows that the losartan remains in the amorphous form over time.

### EXAMPLE 4

100 gm of crystalline losartan potassium and 75 gm of polyvinylpyrrolidone K-30 were dissolved in 1000 ml of water. The solution obtained was dried at 135° C. at a spray rate of 5.0 ml/min and at a pressure of 1.5 kg/cm² in a spray drier for about 30 minutes. The amorphous solid was obtained by co-precipitation.

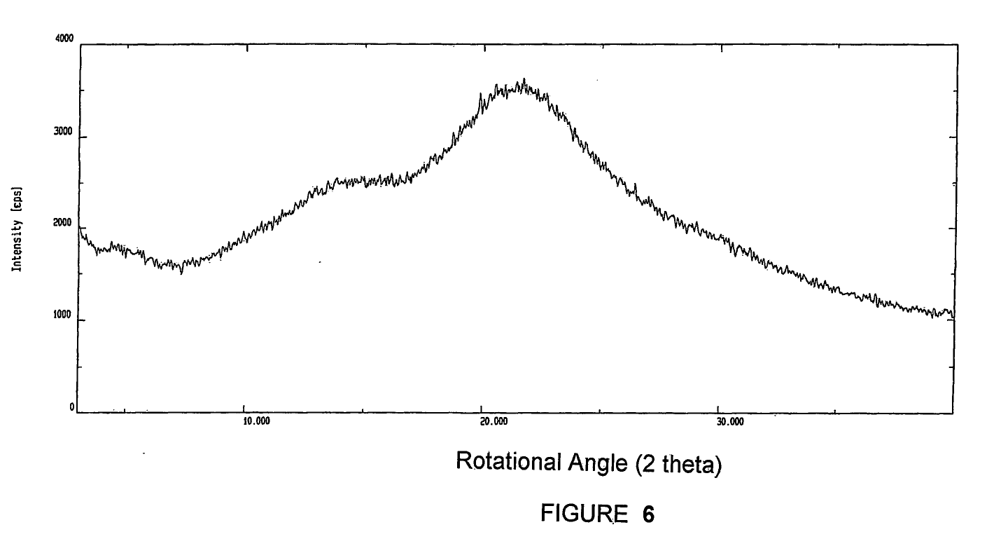

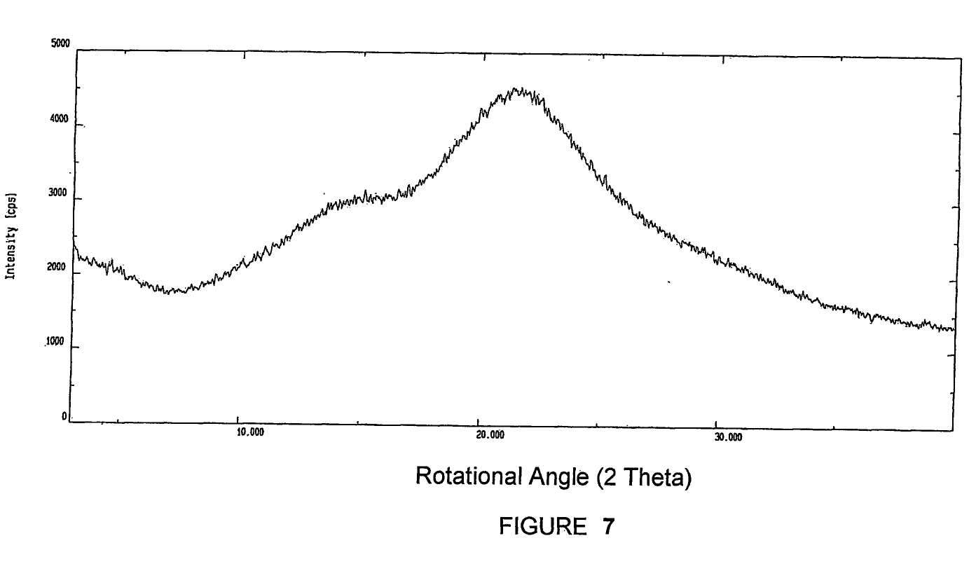

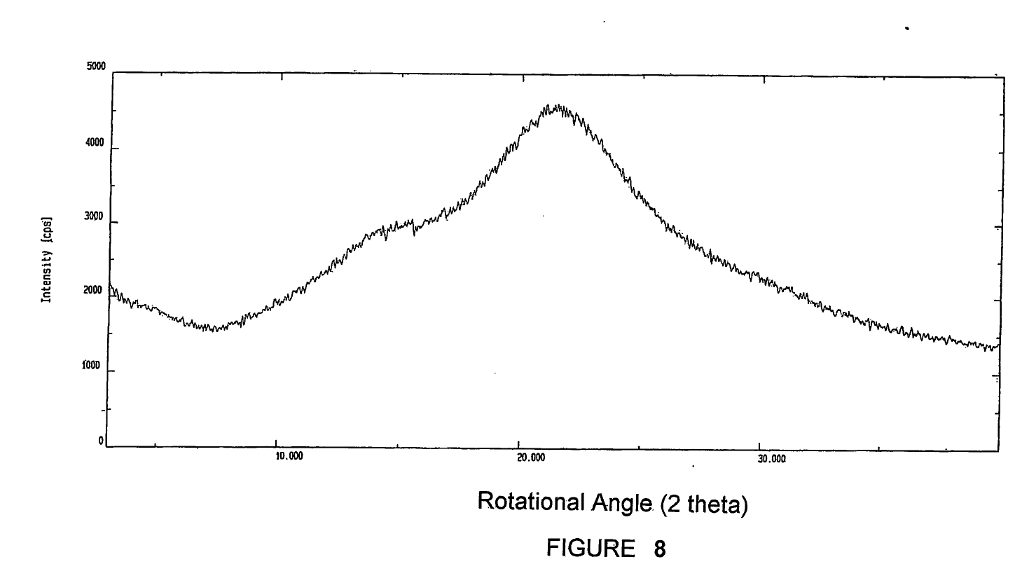

The amorphous, co-precipitated solid obtained was subjected to accelerated stability conditions at 40° C. and 75% relative humidity for three months. It remained in the amorphous form for the three months of accelerated stability testing as evident from the XRD spectra in FIGS. 6-8. The spectra of FIG. 6 is that of the spray dried, co-precipitated mix as measured initially after co-precipitation. This spectra shows the amorphous form of losartan. The spectra of FIG. 7 is that of the spray dried, co-precipitated mix as measured after one month of storage at the accelerated stability conditions (i.e., 40° C. and 75% relative humidity). This spectra shows that the losartan remains in the amorphous form. The spectra of FIG. 8 is that of the spray dried, co-precipitated mix after three months of storage at the accelerated stability condition. Again, this spectra shows that the losartan remains in the amorphous form over time.

### EXAMPLE 5

100 gm of crystalline losartan potassium and 100 gm of polyvinylpyrrolidone K-30 were dissolved in 1000 ml of water. The solution obtained was dried and co-precipitated at 135° C. at a spray rate of 5.0 ml/min and at a pressure of 1.5 kg/cm² in a spray drier for about 40 minutes. The amorphous solid was obtained.

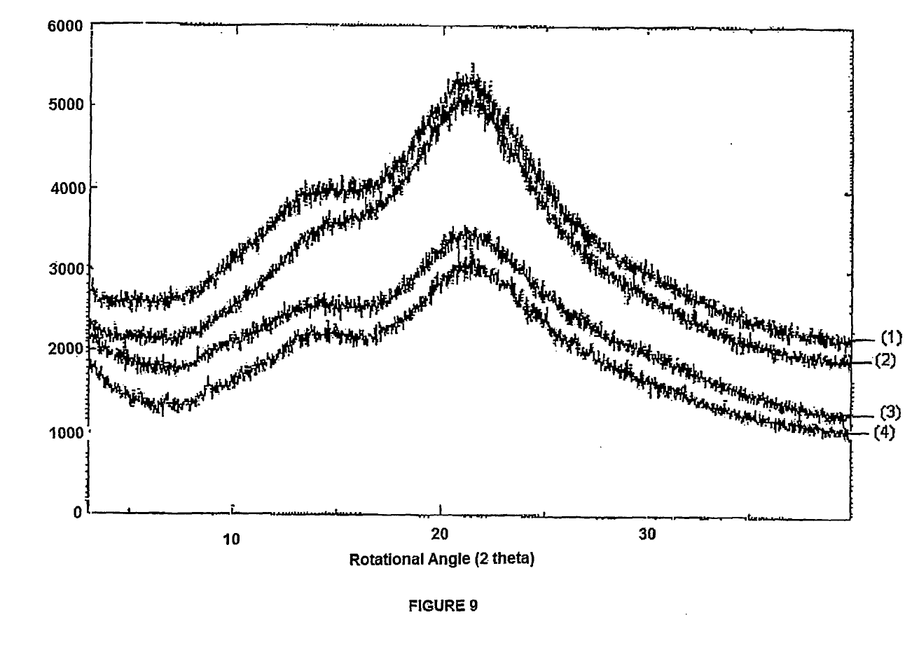

When the amorphous solid obtained was subjected to accelerated stability conditions at 40° C. and 75% relative humidity, it remained in the amorphous form as evident from the XRD spectra in FIG. 9. In FIG. 9, the spectra (1) is that of the spray dried, co-precipitated mix as measured initially, spectra (2) is that of the material taken from the side of the vessel in which the co-precipitation occurs, spectra (3) is that of the spray dried, co-precipitated mix after 24 hours storage at room temperature, and spectra (4) is that of the spray dried, co-precipitated mix after 24 hours storage at 40° C. and 75% relative humidity.

### EXAMPLE 6

150 mg of the amorphous solid (spray dried, co-precipitated mix of losartan potassium and polyvinylpyrrolidone) obtained in Example 2 were blended with 190 mg of anhydrous lactose, 35 mg of microcrystalline cellulose and 20 mg of crosscarmellose sodium. 50 mg of colloidal silicon dioxide were then mixed with the above blend followed by mixing with 7.5 mg of magnesium stearate. The final mixture was then directly compressed into tablets and coated with opadry until a weight gain of 4% was obtained.

### EXAMPLE 7

200 mg of the amorphous solid (spray dried, co-precipitated mix of losartan potassium and PVP) obtained in Example 5 were blended with 230 mg of anhydrous lactose, 40 mg of microcrystalline cellulose and 15 mg of crosscarmellose sodium. 50 mg of colloidal silicon dioxide were then mixed with the above blend and then mixed with 7.5 mg of magnesium stearate. The final mixture was then directly compressed into tablets and coated with opadry until a weight gain of 4% was obtained.

The above examples illustrate that the co-precipitation processes described herein provide stable amorphous losartan that surprisingly does not convert to its crystalline form when stored at 40° C. and 75% relative humidity for three months. The examples also show that dosage forms can be made of the stable amorphous losartan.

While several particular forms of the invention have been illustrated and described, it will be apparent that various modifications and combinations of the invention detailed in the text can be made without departing from the spirit and scope of the invention. For example, all the working examples involve the use of PVP K-30 as the hydrophilic polymer for the preparation of amorphous losartan other grades of PVP as well as other hydrophilic polymers will function in a similar manner with only slight modifications to a few parameters in most cases. Further, it is contemplated that any single feature or any combination of optional features of the inventive variations described herein may be specifically excluded from the claimed invention and be so described as a negative limitation. Accordingly, it is not intended that the invention be limited, except as by the appended claims.

We claim:

1. A process for stabilizing amorphous losartan, the process comprising:
preparing an aqueous solution of losartan and one or more hydrophilic polymers; and
spray drying the aqueous solution of losartan and one or more hydrophilic polymers to form a mixture, whereby the amorphous losartan and one or more hydrophilic polymers are co-precipitated from the aqueous solution.

2. The process according to claim 1, wherein the aqueous solution is prepared in an aqueous solvent selected from the group consisting of water, water miscible solvents and mixtures thereof.

3. The process according to claim 2, wherein the aqueous solvent comprises water.

4. The process according to claim 2, wherein the water miscible solvent comprises one or more of methanol, ethanol, n-propanol and isopropanol.

5. The process according to claim 1, wherein the hydrophilic polymer comprise one or more of polyvinylpyrrolidone (PVP), polyvinyl alcohol, hydroxypropyl methylcellulose, methylcellulose, carboxymethyl cellulose, sodium carboxymethylcellulose, hydroxyethylcellulose, polyvinyl acetate, carbopols and combinations thereof.

6. The process according to claim 5, wherein the hydrophilic polymer comprises polyvinylpyrrolidone.

7. The process according to claim 6, wherein the polyvinylpyrrolidone comprises one or more of the grades PVP K-15, K-25, K-30, K-60 and K-90.

8. The process according to claim 7, wherein the polyvinylpyrrolidone grade comprises PVP K-30.

9. The process according to claim 6, wherein the ratio of polyvinylpyrrolidone to losartan is from about 0.5:1 to about 1:1.5.

10. The process according to claim 9, wherein the ratio of polyvinylpyrrolidone to losartan is 1:1.

11. The process according to claim 5, wherein the hydrophilic polymer comprises polyvinyl alcohol.

12. The process according to claim 5, wherein the hydrophilic polymer comprises hydroxypropyl methylcellulose.

13. The process according to claim 1, wherein the spray drying is carried out at a temperature of more than about 60° C.

14. The process according to claim 13, wherein the spray drying is carried out at a temperature of about 135° C.

15. The process according to claim 1, further comprising processing the mixture with one or more pharmaceutically inert excipients.

16. The process according to claim 15, wherein the pharmaceutically inert excipient comprises one or more diluents, binders, disintegrants, coloring agents, flavoring agents, stabilizers, surfactants, lubricants, glidants, plasticizers, and preservatives.

17. The process according to claim 15, further comprising forming one or more of a tablet, a capsule, and a powder.

18. The process according to claim 1, wherein the losartan remains amorphous as measured by X ray diffraction after accelerated stability testing at 40° C. and 75% relative humidity for three months.

19. A pharmaceutical composition comprising:
a co-precipitated mixture of amorphous losartan and one or more hydrophilic polymers.

20. The pharmaceutical composition of claim 19, wherein the losartan remains amorphous as measured by X ray diffraction after accelerated stability testing at 40° C. and 75% relative humidity for three months.

21. The pharmaceutical composition according to claim 19, wherein the hydrophilic polymer comprise one or more of polyvinylpyrrolidone (PVP), polyvinyl alcohol, hydroxypropyl methylcellulose, methylcellulose, carboxymethyl cellulose, sodium carboxymethylcellulose, hydroxyethylcellulose, polyvinyl acetate, carbopols and combinations thereof.

22. The pharmaceutical composition according to claim 21, wherein the hydrophilic polymer comprises polyvinylpyrrolidone.

23. The pharmaceutical composition of claim 19, wherein the composition further comprises one or more pharmaceutically inert excipients.

24. The pharmaceutical composition according to claim 23, wherein the one or more pharmaceutically inert excipients comprise one or more of diluents, binders, disintegrants, coloring agents, flavoring agents, stabilizers, surfactants, lubricants, glidants, plasticizers and preservatives.

25. The pharmaceutical composition according to claim 19, wherein the pharmaceutical composition is a solid dosage form.

26. The pharmaceutical composition to claim 25, wherein the solid dosage form comprises one or more of tablets, capsules, and powders.

27. The pharmaceutical composition according to claim 26, wherein the solid dosage form comprises a tablet.

28. The pharmaceutical composition according to claim 19, wherein a ratio of polyvinylpyrrolidone to losartan is from about 0.5:1 to about 1:1.5.

29. The pharmaceutical composition according to claim 28, wherein the ratio of polyvinylpyrrolidone to losartan is 1:1.

30. A method for the treatment of angiotensin-induced hypertension in a mammal, the method comprising administering a pharmaceutical composition comprising a co-precipitate of amorphous losartan and one or more hydrophilic polymers, and one or more pharmaceutically inert excipients.

31. The method according to claim 30, wherein the hydrophilic polymer comprises polyvinylpyrrolidone.

32. The method according to claim 31, wherein the polyvinylpyrrolidone comprises one or more of the grades PVP K-15, K-25, K-30, K-60 and K-90.

33. The method according to claim 31, wherein the polyvinylpyrtolidone grade comprises PVP K-30.

34. The method according to claim 30, wherein a ratio of polyvinylpyrrolidone to losartan is from about 0.5:1 to about 1:1.5.

35. The method according to claim 30, wherein the ratio of polyvinylpyrrolidone to losartan is 1:1.

36. The method of claim 30, wherein the losartan remains amorphous as measured by X ray diffraction after accelerated stability testing at 40° C. and 75% relative humidity for three months.

37. A co-precipitate of amorphous losartan and one or more hydrophilic polymers.

38. The co-precipitate of claim 37, wherein a ratio of hydrophilic polymer to losartan is from about 0.5:1 to about 1:1.5.

39. The co-precipitate of claim 37, wherein the ratio of hydrophilic polymer to losartan is 1:1.
